(19) United States
(12) Patent Application Publication
(10) Pub. No.: US 2020/0289334 A1
(43) Pub. Date: Sep. 17, 2020

(54) MEDICAL BANDAGE WITH SUPPORT AND METHOD OF USING THE SAME

(71) Applicant: CONCEPTO, LLC, Lebanon, OH (US)

(72) Inventors: Sambhu N. Choudhury, Cincinnati, OH (US); Sean M. Lynch, Cincinnati, OH (US); Arturo David Sanchez, Lebanon, OH (US); Eric G. Holcomb, Cincinnati (US)

(73) Assignee: CONCEPTO, LLC, Lebanon, OH (US)

(21) Appl. No.: 16/299,788

(22) Filed: Mar. 12, 2019

(51) Int. Cl. A61F 13/10; A61B 46/27; A61F 13/60; A61F 13/82

(52) U.S. Cl. CPC A61F 13/10; A61B 46/27; A61B 2046/205; A61F 13/82; A61F 13/60

(57) ABSTRACT

A medical bandage includes a bandage substrate having a portion designated to receive and underlie an arm of a patient as a non-sterile area. A flap attached longitudinally along one edge of the non-sterile area of the bandage substrate is positionable in: (i) an open position for insertion of the arm; and (ii) a closed position over top of the arm of the patient. The flap has an opening that exposes at least a forearm portion as a sterilizable area of the arm of the patient for therapeutic or diagnostic access to a blood vein. An inner adhesive layer is attached to an inner surface of the flap around the opening to releasably adhere to the arm of the patient. An access panel is sized to cover the opening. An outer adhesive layer attached to an outer surface releasably adheres to an outer annular area of the access panel.

### CLAIM OF PRIORITY UNDER 35 U.S.C. § 120

The present Application for Patent is a continuation-in-part of patent application Ser. No. 14/798,278, entitled “MEDICAL BANDAGE WITH SUPPORT AND METHOD OF USING THE SAME” filed Jul. 13, 2015, pending, which is a continuation-in-part of patent application Ser. No. 13/224,146 entitled “MEDICAL BANDAGE WITH SUPPORT AND METHOD OF USING THE SAME” filed Sep. 1, 2011, now abandoned, and claiming priority to provisional U.S. Patent Application No. 61/379,062 as filed on Sep. 1, 2010, each of which is assigned to the assignee hereof and hereby expressly incorporated by reference herein.

### FIELD OF THE INVENTION

The present disclosure relates generally to a medical bandage, and more particularly to a medical bandage with an extendable sleeve to create a substantially sterile field and a method of using the same medical bandage.

### BACKGROUND

Rapid care is foremost in the initial treatment and rehabilitation of individuals with open wounds stemming from missile trauma, open fractures, and bleeding extremities, among other injuries. Because primary wound closure is not usually done on the battlefield, transport allows for final definitive care of the individual. Further, the extent of trauma in the extremities that are presented for treatment is more significant and complicated than in previous conflicts. Thus, the ability to contain a wound and rapidly transport an individual from the battlefield to the hospital is critical. However, hemorrhage, infection, and other types of wound complication remain major obstacles for providing proper wound care.

Gauze dressings are typically used for immediate coverage of wounds. Usually, gauze dressings absorb some of the blood from the open wound and allow for an immediate barrier for the soft tissues. This allows for compression of a bleeding appendage and often for hemostasis due to clot formation. To apply gauze dressing, packs of sterilized gauze are opened in the field and then applied to an affected appendage with tape or a wrap. However, multiple packs are often required making the process more cumbersome and less efficient. Further, the gauze is permeable, and even if it is applied in multiple layers, it often does not prevent seepage through the dressing. These problems can lead to wound contamination, and also, cross-contamination and/or contamination of health care workers.

Plastic dressings or tape may be applied to prevent additional seepage. For example, sticky “cover-all” dressings can be applied as a barrier. While these can help to prevent fluid loss and contamination, the necessary adhesives can also create additional trauma and pain around the wound.

Traumatized extremities are currently controlled with dressings. Dressing wrap is used to apply pressure to bleeding areas of the wound, form a substrate for clot formation, decrease contamination from the field, prevent contamination of transport vehicles and carts, provide protection from additional trauma, and to quantify the amount of blood loss.

Unfortunately, it often takes a medically trained technician to apply dressings appropriately with the proper, sterile technique. Even for medically trained technicians, however, applying dressings can be challenging. For example, gauze must be removed from often-bulky packs and kept sterile until applied to the appendage. If there is a broad expanse of limb that needs to be covered, an individual often has difficulty applying multiple gauzes to an appendage and holding them in place. Any type of adhesive carries the risk of damaging adjacent skin and tissue and wrapping an injured appendage can often be painful because of compromised soft tissues or broken bones. Further, in a military setting, applying dressings to an individual without contaminating the wound or causing significant additional injury can be extremely difficult. And regardless of the dressing type, once the gauze dressings become saturated, blood may leak around the edges of the wound and through the dressing itself.

In addition to applying the proper covering, a sterile field is required in order to control the bacterial content surrounding a surgical wound and deter infection. Additionally, the appendage must be sterilized with antiseptic prior to creating any cover for the limb. However, in a trauma or emergency setting the ability to create and maintain a sterile field is difficult and often unwieldy and frustrating. Further, the coverage is often not circumferential. And the chances of contamination are even greater if the individual is moved or if there is debris in the area.

What is needed is a device that allows for the rapid containment of an appendage, reduces blood and body fluid exposure and contamination, is easily transported, allows for a circumferential sterile field, decreases transport time and complications, and can be ergonomically, efficiently, and safely applied by non-medical personnel.

### SUMMARY OF THE INVENTION

Various exemplary embodiments of the present innovation provide a medical bandage that includes a bandage substrate having a portion designated to receive and underlie an arm of a patient as a non-sterile area. A flap is attached longitudinally along one edge of the non-sterile area of the bandage substrate and is positionable in: (i) an open position for insertion of the arm of the patient; and (ii) a closed position over top of the arm of the patient. The flap has an opening that exposes at least a forearm portion as a sterilizable area of the arm of the patient for therapeutic or diagnostic access to a blood vein. An inner adhesive layer is attached to an inner surface of the flap around the opening to releasably adhere to the arm of the patient. An access panel is sized to cover the opening. An outer adhesive layer is attached to an outer surface of the flap around the opening to releasably adhere to an outer annular area of the access panel.

In one aspect of the present disclosure, a medical bandage includes a bandage substrate having a first portion designated to receive and underlie an arm of a patient as a non-sterile area. The bandage substrate has a second portion that wraps over a forearm between a wrist and an inner side of the elbow of the patient. The second portion has an opening that exposes at least a portion of the forearm for accessing a blood vein. An inner adhesive layer is attached around a periphery of the second portion around the opening to adhere to the forearm of the patient. When adhered, the inner adhesive layer separates an unsterilized portion of the patient's arm from a sterilized portion of the patient's arm exposed through the opening. An outer protective cover is releasably engaged to the close the opening.

These and other features are explained more fully in the embodiments illustrated below. It should be understood that in general the features of one embodiment also may be used in combination with features of another embodiment and that the embodiments are not intended to limit the scope of the invention.

### BRIEF DESCRIPTION OF THE DRAWINGS

The various exemplary embodiments of the present innovation, which will become more apparent as the description proceeds, are described in the following detailed description in conjunction with the accompanying drawings, in which:

FIG. 1 illustrates an isometric depiction of a medical bandage encompassing an appendage of a patient, according to one or more embodiments;

FIG. 2A illustrates a side view of the medical bandage of FIG. 1 in an initial, untightened state, according to one or more embodiments;

FIG. 2B illustrates a side view of the medical bandage of FIG. 2A in tightened state, according to one or more embodiments;

FIG. 3 illustrates an isometric view of an example medical bandage with a band structure, according to one or more embodiments;

FIGS. 4A-4C illustrate an end view of the band structure of the medical bandage of FIG. 3 being opened, according to one or more embodiments;

FIG. 5 illustrated an isometric view of the medical bandage of FIG. 3, according to one or more embodiments;

FIG. 6 illustrates a cross-sectional view of the medical bandage of FIG. 3, according to one or more embodiments;

FIG. 7 illustrates an isometric view of the medical bandage of FIG. 3 as initially inserted over an arm of the patient, according to one or more embodiments;

FIG. 8 illustrates an isometric view of the medical bandage of FIG. 7 that includes a distal band, according to one or more embodiments;

FIG. 9 illustrates a depiction of an extendible sleeve is in the form of a sheet enclosing an appendage, according to one or more embodiments;

FIG. 10 illustrates an extendible sleeve in a folded state within a band, according to one or more embodiments;

FIG. 11 illustrates the extendible sleeve of FIG. 10 in an unfolded state outside of the band, according to one or more embodiments;

FIG. 12 illustrates an extendible sleeve is in the form of a sheet prior to being secured around an appendage, according to one or more embodiments;

FIG. 13 illustrates an isometric view of a patient wearing a medical bandage with a sterilizable and accessible forearm window, according to one or more embodiments;

FIG. 14 illustrates a side cross-sectional view of the forearm window of the medical bandage having internal and external cover panels of FIG. 13, according to one or more embodiments;

FIG. 15 illustrates a medical bandage laid open and receiving an arm of a patient and with an internal cover panel removed to expose an adhesive around a forearm access opening, according to one or more embodiments;

FIG. 16 illustrates the medical bandage of FIG. 15 with a window opening placed over the forearm of the patient, according to one or more embodiments;

FIG. 17 illustrates an isometric view of the patient wearing the medical bandage of FIG. 16 with an outer protective cover removed to expose the forearm, according to one or more embodiments;

FIG. 18 illustrates a top view of an example single-access forearm medical bandage in an open configuration, according to one or more embodiment;

FIG. 19 illustrates a top view of the example single-access forearm medical bandage in a closed configuration;

FIG. 20 illustrates a top view of an example double-access forearm medical bandage in a closed configuration, according to one or more embodiments;

FIG. 21 illustrates a folded medical bandage, according to one or more embodiments;

FIG. 22 illustrates an unfolded medical bandage, according to one or more embodiments;

FIG. 23 illustrates the unfolded medical bandage with a shoulder opening, according to one or more embodiments; and

FIG. 24 illustrates a forearm portion of the unfolded medical bandage, according to one or more embodiments;

FIG. 25 illustrates a forearm placed on the forearm portion of the unfolded medical bandage, according to one or more embodiments; and

FIG. 26 illustrates the forearm covered by the medical bandage, according to one or more embodiments.

### DETAILED DESCRIPTION

Various exemplary embodiments of the present innovation include a medical bandage. The medical bandage comprises a band having an extendable sleeve. The medical bandage also comprises a first strap, wherein the first strap is positioned on a top portion of the band, and a second strap, wherein the second strap is positioned on a bottom portion of the extendable sleeve.

In one aspect of the present innovation, a medical bandage includes a bag-type container having a mouth defined by at least one elongate, annular panel to longitudinally encompass an appendage of a patient. An adhesive layer is disposed on an exterior portion of the bag-type container. A removable backing layer is selectively removable from the adhesive layer to expose the adhesive layer. A portion of the bag-time container is foldable onto the exposed adhesive layer to fixedly reduce a volume of the bag-type container to conform to an appendage of a patient.

Various exemplary embodiments of the present innovation include a method of using a medical bandage. The method comprises the steps of positioning the medical bandage on an appendage of an individual, wherein the medical bandage comprises a band having an extendable sleeve. The medical bandage also comprises a first strap, wherein the first strap is positioned on a top portion of the band, and a second strap, wherein the second strap is positioned on a bottom portion of the extendable sleeve. The method also comprises the steps of tightening the first strap to secure the top portion of the band to the appendage. The method further comprises the steps of stretching the extendable sleeve over the wound. The method also comprises the steps of tightening the second strap to secure the bottom portion of the extendable sleeve to the appendage.

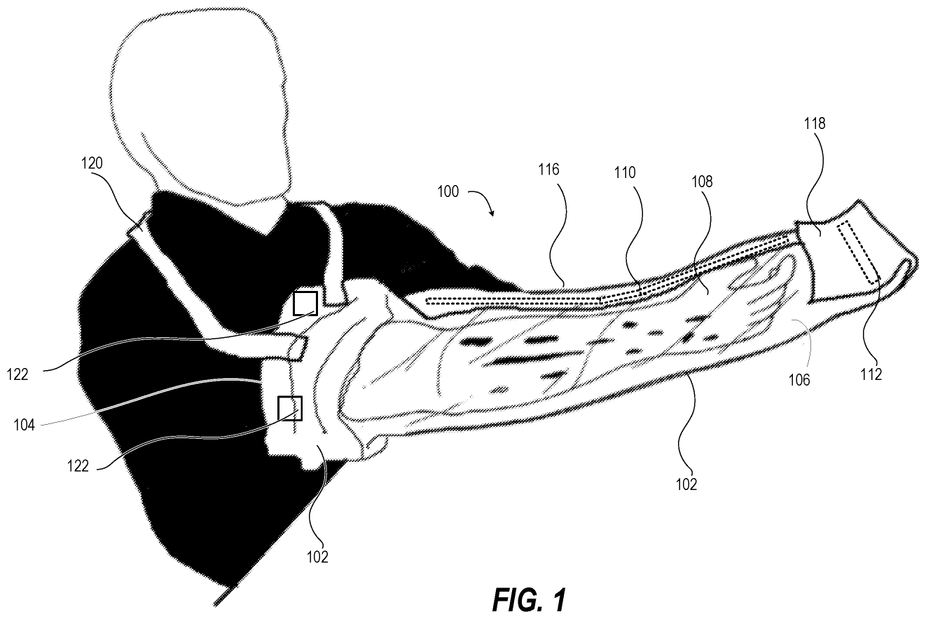

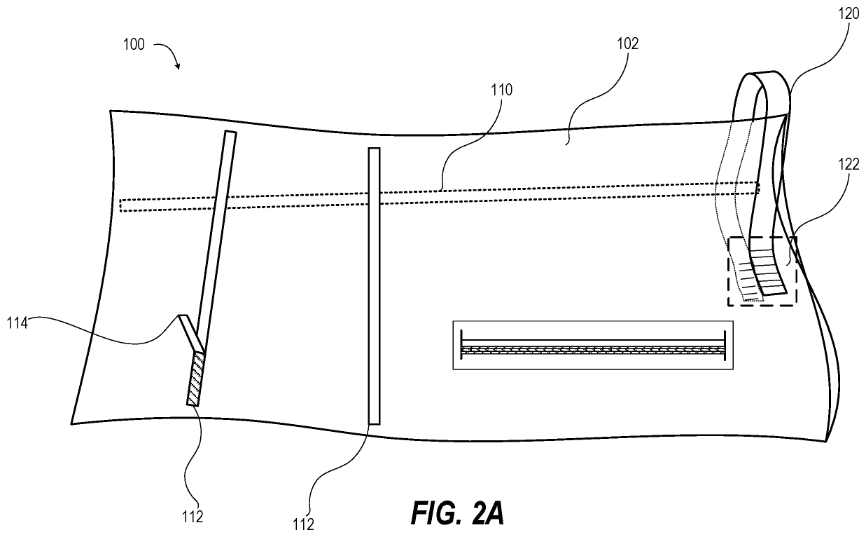

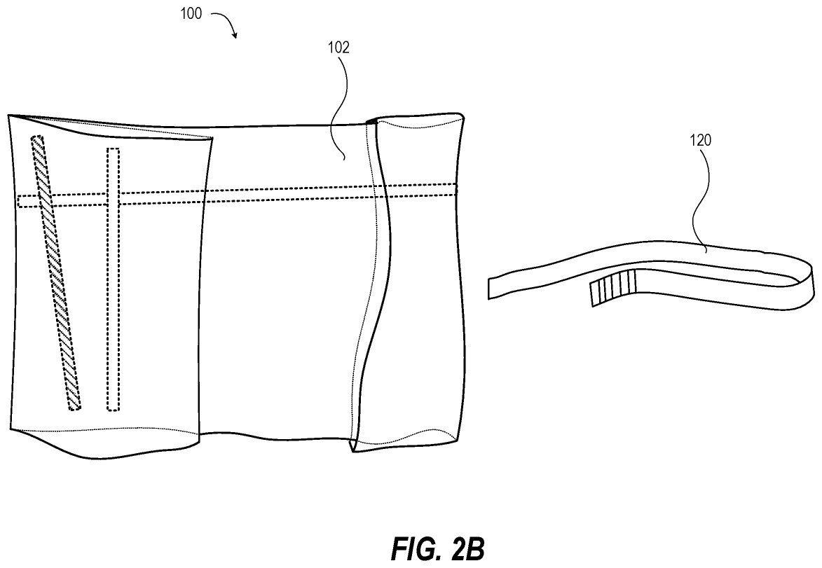

In one or more embodiments, FIG. 1 illustrates a medical bandage 100 that includes a bag-type container 102 having a mouth 104 defined by at least one elongate, annular panel 106 to longitudinally encompass an appendage 108 of a patient. The mouth 104 is annularly rolled up in a first example. At least one adhesive layer is disposed on an exterior portion of the bag-type container 102. In one embodiment, a longitudinal adhesive layer 110 and a distal lateral adhesive layer 112 are provided, which are hidden beneath backing layer strips 114 (FIG. 2A) until ready for use. FIG. 2B illustrates a lateral flap portion 116 folded onto the longitudinal adhesive layer 110 to reduce a lateral dimension of the bag-type container 102. A distal flap portion 118 of the bag-time container 102 is folded onto the exposed distal adhesive layer 112 to reduce a longitudinal dimension of the bag-type container 102. Both adjustments fixedly reduce a volume of the bag-type container 102 to conform to the appendage 108 of a patient.

To assist in holding the medical bandage 100 onto the appendage 108 of the patient, a strap 120 is provided. Two outwardly disposed backing layers 122 are attached to the bag-type container to temporarily store the strap 120. In particular, the strap 120 includes adhesive layers on respective ends that are releasably engaged to the two outwardly disposed backing layers 122 for selectively removal, positioning around the patient, and reattaching to the bag-type container 102. In one embodiment, the bag-type container 102 can include opposed side panels 124 comprise a transparent, fluid impermeable material for visual inspection without exposing the appendage 108 to external contaminants. FIG. 2A illustrates the medical bandage 100 in an initial state. FIG. 2B illustrates the medical bandage in a folded state.

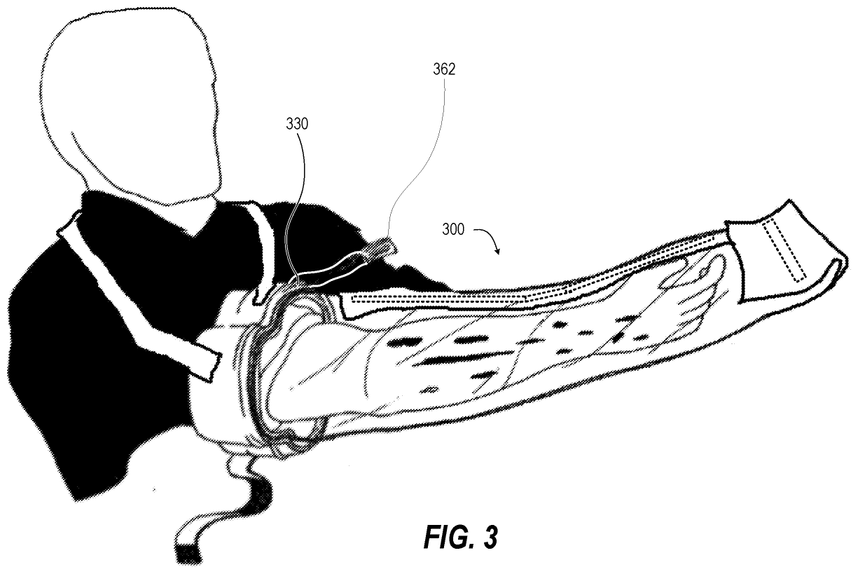

FIG. 3 illustrates a medical bandage 300 that further includes a cuff or band structure 330 that includes an annular compartment that contains the bag-like container in a longitudinally compressed state and with the distal end open, wherein one of a lateral adhesive layer and a distal strap closes a distal end of the bag-like container to itself of a distal portion of the appendage to form an enclosure about an injured portion of the appendage. In an exemplary embodiment of the present invention, the medical bandage further comprises a tourniquet 362, wherein the tourniquet is positioned on or adjacent to the band 330.

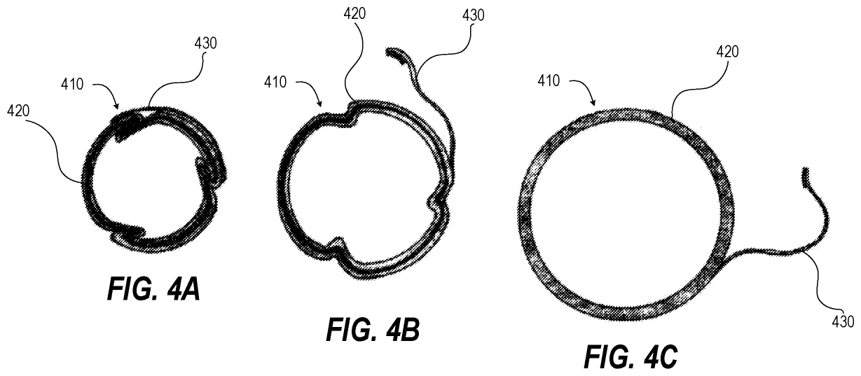

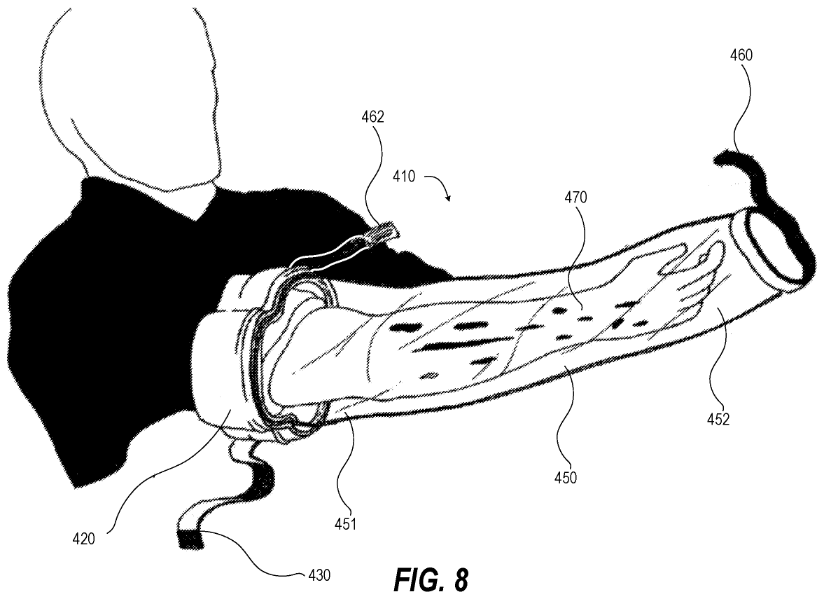

FIGS. 4A-4C and 5-8 illustrate a medical bandage according to one or more exemplary embodiments of the present invention. The medical bandage 410 comprises a band 420 having an extendable sleeve 450. The medical bandage also comprises a first strap 430, wherein the first strap connected to a first end 451 of the extendible sleeve 450, and a second strap 460, wherein the second strap 460 is connected to a second end 452 of the extendable sleeve 450. In an exemplary embodiment of the present invention, the medical bandage further comprises a tourniquet 462 (FIG. 8), wherein the tourniquet is positioned on or adjacent to the band 420.

FIGS. 4A-4C illustrate the medical band of exemplary embodiments of the present invention as the medical bandage is unfolded to fit around an appendage, i.e., an arm or a leg, of an individual. In an exemplary embodiment of the present invention, the medical bandage is delivered in a sterile package. Once the package is opened, the medical bandage, shaped like a doughnut, is unfolded to fit around the appendage of the individual.

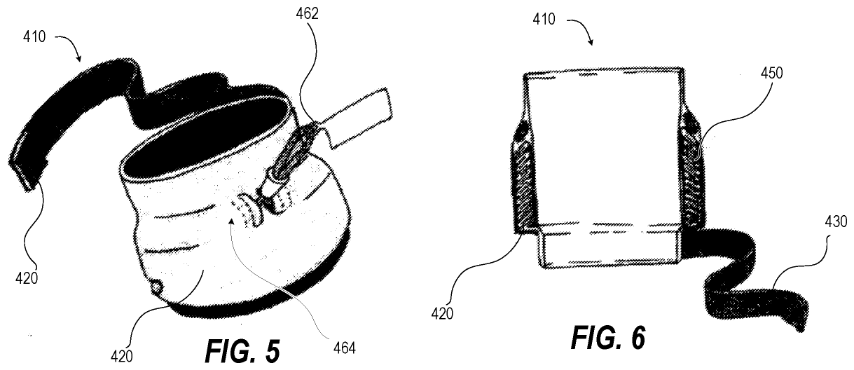

FIG. 5 illustrates the medical bandage with the first strap. In an exemplary embodiment of the present invention, the first strap connects to the band by hooks and fasteners, thread, adhesives, or the like. In exemplary embodiments of the present invention, the first strap can be fitted and tightened by snaps, buttons, hooks, and the like. FIG. 5 also illustrates the tourniquet. In an exemplary embodiment of the present invention, the tourniquet 462 is embedded within the band. In an exemplary embodiment of the present invention, the tourniquet 462 can be inflated with a pump included with the sleeve and adjusted by a tourniquet gauge 464.

FIG. 6 illustrates a cross-sectional view of the medical bandage. In an exemplary embodiment of the present invention, the extendable sleeve is packaged such that it is tucked into a chamber within the band. The second strap is positioned at the bottom portion of the extendable sleeve. In an exemplary embodiment of the present invention, the second strap can be fitted and tightened by snaps, buttons, hooks, and the like.

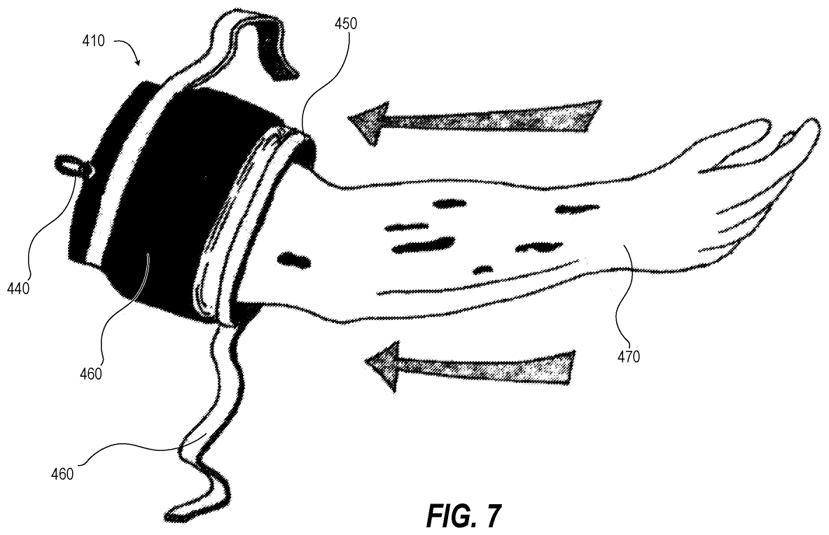

The present invention also relates to a method for using the medical bandage. In an exemplary embodiment of the present invention, non-medical personnel may apply the medical bandage. The medical bandage is positioned around an appendage of an individual and moved in a direction towards the axial trunk of the individual as displayed in FIG. 7. In an exemplary embodiment of the present invention, a wound on the appendage to be enclosed by the present invention is sterilized before sliding the medical bandage over the appendage. The first strap is tightened to secure the first end of the extendible sleeve, preferably still encased in the band, to the appendage. The extendible sleeve is preferably still in a compact state within the band.

Once the band is at a desired location and secured by the first strap, the extendable sleeve removed from the band and stretched over the wound on the appendage, as displayed in FIG. 8. Once the extendable sleeve is at a desired length, the second end of the extendible sleeve is secured to the appendage via the second strap. In an exemplary embodiment of the present invention, once the second strap is secured, a sterile field should have been created within the medical bandage. In an exemplary embodiment of the present invention, air may be evacuated from the sterile field via a drain port (not shown).

Further, in an exemplary embodiment of the present innovation, the extendable sleeve may be opened to allow for irrigation. In an exemplary embodiment of the present innovation, the drain port allows for removal of bodily fluids, excess medicines applied, and contaminants.

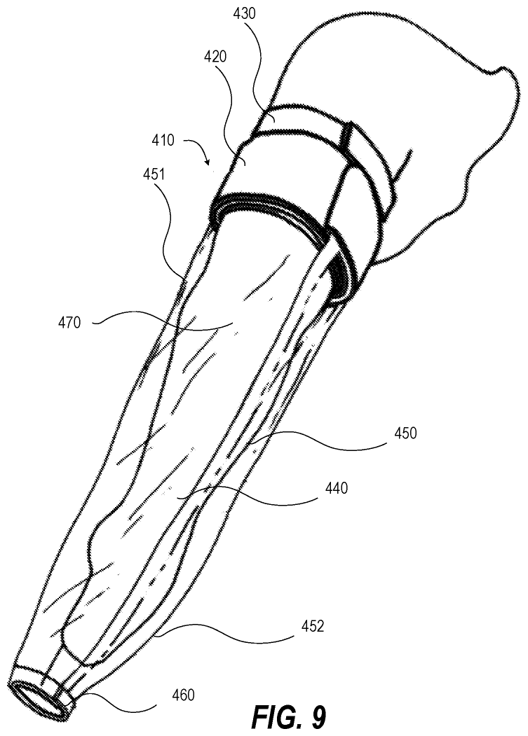

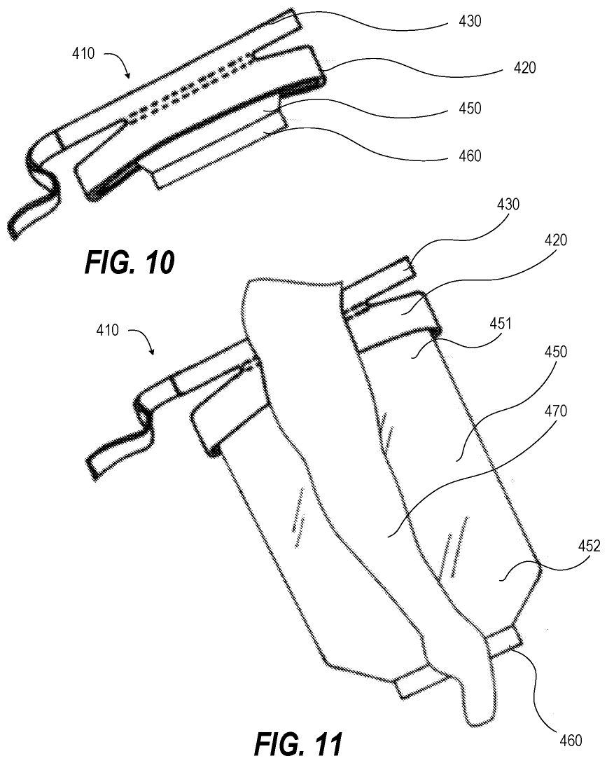

In various exemplary embodiments, the extendable sleeve is in the form of a sheet when expanded from the band. That is, the sheet has an opening substantially between the band and the second strap. Such variation of the extendible sleeve is illustrated in FIGS. 9-12.

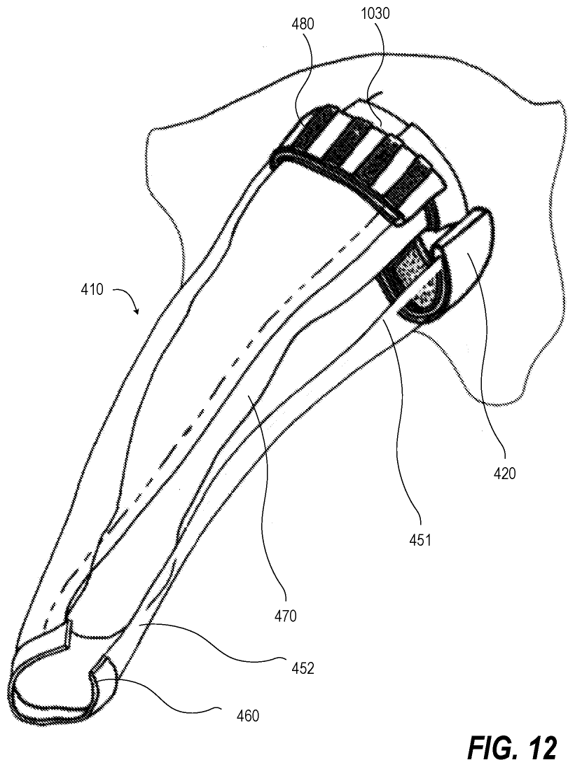

When the extendable sleeve is in the form of a sheet, the extendible sleeve is in a substantially folded form that can be opened to allow the appendage to be placed upon the open extendible sleeve. The extendable sleeve is then placed around the appendage and secured via one or more securing means, which may be in the form of, for example, hooks and fasteners, one or more adhesives, snaps, buttons, or a combination thereof, in order to contain the wound on the appendage. The securing means may also be incorporated into the band as illustrated in FIG. 12. Additional strap and/or compression bandages may be utilized in conjunction with the present innovation to compress and secure the sleeve to the appendage.

It is exemplary that the extendible sleeve is transparent to allow for visual scrutiny and on-going observation of the enclosed appendage until such time as advanced medical attention may be available to aid in healing the afflicted appendage.

Dressings may be applied after the medical bandage has been applied to the appendage. In an exemplary embodiment of the present innovation, procedures are performed on the wound through one or more closable openings created in the medical bandage. In an exemplary embodiment, a closable opening may be present running in a direction of the first end to the second end of the extendible sleeve. The extendible sleeve, if opened, may be resealed and/or wrapped with tape, one or more adhesives, hooks and fasteners, or a combination thereof. In an exemplary embodiment of the present innovation, a splint may be applied over the medical bandage to immobilize the appendage.

In another exemplary embodiment of the present innovation, heat or cool packs may be applied over the medical bandage to avoid direct contact with the wound of the afflicted appendage.

In an exemplary embodiment of the present innovation, the medical bandage can be presented in multiple sizes and for different types of appendages. In an exemplary embodiment of the present innovation, the medical bandage is a long roll that encloses and attached to the appendage.

A tourniquet, when present, is preferably positioned on or adjacent to the band at the first end of the extendible sleeve. The tourniquet may be used to slow or stop blood flow to the appendage enclosed by the extendible sleeve. The tourniquet may be adjusted via a pump and adjusted via a tourniquet gauge (not shown).

The invention also features a method of reducing inflammation at a site in a mammal in need thereof by implanting an article of the invention at the site, wherein the article includes an anti-inflammatory agent which is released from the surface of the article in response to selective hydrolysis by peptidases expressed during inflammation in an amount sufficient to reduce inflammation.

The invention also features a method of reducing restenosis at a site in a mammal in need thereof by implanting an article of the invention at the site, wherein the article includes an anti-proliferative agent which is released from the surface of the article in response to selective hydrolysis by peptidases expressed during proliferation in an amount sufficient to reduce restenosis.

In another aspect, the invention features a method of reducing pain at a site in a mammal in need thereof by implanting an article of the invention at the site, wherein the article includes an analgesic or anesthetic agent which is released from the surface of the article in response to selective hydrolysis by peptidases expressed during pain in an amount sufficient to reduce pain.

In still another aspect, the invention features a method of relaxing muscle at a site in a mammal in need thereof by implanting an article of the invention at the site, wherein the article includes an antispasmodic agent which is released from the surface of the article in response to selective hydrolysis by peptidases expressed during muscle spasm in an amount sufficient to relax muscle.

While this invention has been described in conjunction with the specific embodiments outlined above, it is evident that many alternatives, modifications and variations will be apparent to those skilled in the art. Accordingly, the exemplary embodiments of the invention as set forth above are intended to be illustrative, not limiting. Various changes may be made without departing from the spirit and scope of the invention.

In one or more exemplary embodiments, a method of bandaging a wounded appendage of an individual can include (a) examining the wound on the appendage of the individual; (b) placing a medical bandage to a position on the appendage of the individual; (c) tightening the first strap against the appendage of the individual; (d) extending the extendable sleeve from the band and in a direction of the wound on the appendage; (e) tightening the second strap against the appendage of the individual once the extendable sleeve is sufficiently and adequately encasing the wound on the appendage; and (f) transporting the individual for advanced medical care. The extendible sleeve can be in a substantially sterile state until just prior to enclosing an appendage. In one or more embodiments, the medical bandage can include a tourniquet at the first end of the extendible sleeve, which can be utilized to slow or stop blood flow to the appendage encased by the extendable sleeve. The extendible sleeve can be substantially transparent for visual scrutiny of the enclosed appendage. The extendible sleeve can include a drain port for drawing off excess bodily fluids, air, contaminants, or medicines from between the appendage and the extendible sleeve. The extendible sleeve can include a closeable opening for accessing the appendage for medical attention, wherein the closeable opening is sealed via one or more fasteners. The extendible sleeve can be secured via the first strap, the second strap, and one or more securing means.

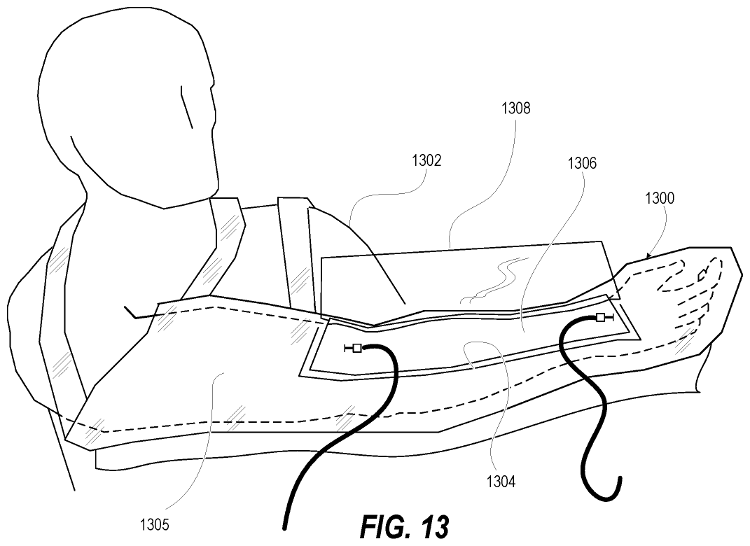

FIG. 13 illustrates an isometric view of a patient wearing a medical bandage 1300 worn by a patient 1302. The medical bandage 1300 has a window opening 1304 formed in a bandage substrate 1306 that exposes at least a portion of an appendage 1308 of the patient and that is selectably closeable with an outer protective cover 1310. In one or more embodiments, the medical bandage 1300 has two or more window openings 1304 formed in a bandage substrate 1306 that exposes at least a portion of an appendage 1308 of the patient and that is selectably closeable with an outer protective cover 1310. In one or more embodiments, the window opening 1304 is made of a tinted, semitransparent, clear, or translucent material that allows the user to see the underlying extremity of body region. In one or more embodiments, the majority, or the entirety of the bandage substrate 1306 is made from a tinted, semi-transparent, or translucent polymer material that is sterile and waterproof. In one or more embodiments, the window opening 1304 is configured such that a needle or medical instrument may penetrate the polymer window to allow immediate access to the underlying extremity of body region.

The polymer material may have antimicrobial characteristics to reduce infection potential and may be left in place post-operatively. In one embodiment, the surface of the material can be coated with any antimicrobial substance to provide the benefit of carrying the sterility of the surgical field through the post-operative healing time. In other embodiments, the antimicrobial characteristic is incorporated into the plastic/incise material. Said another way, the antimicrobial properties of the surgical drape can be due to the surgical drape's material or an antimicrobial substance added to the surgical drape's surface. Antimicrobial substances include, but are not limited to, germicides, antibiotics, antibacterials, antivirals, antifungals, antiprotozoals and antiparasites. Specifically, silver ions or compounds, chlorhexidine, iodine or iodine based, triclosan, bacitracin, clindamycin, erythromycin, metronidazole, mupirocin, neomycin, retamapulin, polymyxin B, mupirocin, fusidic acid, gentamicin, or combinations thereof are contemplated. The antimicrobial substance can be provided in solutions, pastes, suspensions, gels or films. An advantage of this embodiment of the surgical drape is the decrease in the chance of post-operative infection.

Intravenous access to the forearm 1308 is provided through the window opening 1304 In one or more embodiments, the outer protective cover 1308 is a flap attached along one edge to the bandage substrate 1306. In one or more embodiments, the outer protective cover 1310 is transparent for inspecting the forearm 1306.

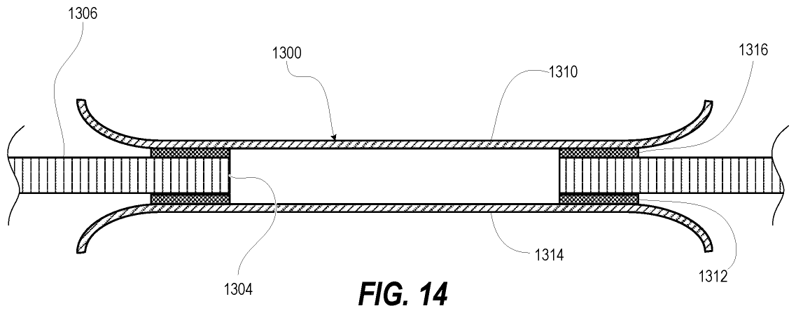

FIG. 14 illustrates the medical bandage 1300 as having an inner adhesive layer 1312 that is attached around a periphery of the window opening 1304 to adhere to the forearm 1306 of the patient 1302 (FIG. 13). The inner adhesive layer 1312 separates an unsterilized portion of the forearm 1306 of the patient 1302 (FIG. 13) from a sterilized portion of the patient's arm exposed through the window opening 1304. Before use, an inner protective cover 1314 is releasably attached to the inner adhesive layer 1312. The medical bandage 1300 has an outer adhesive layer 1316 that releasably engages the outer protective cover 1310.

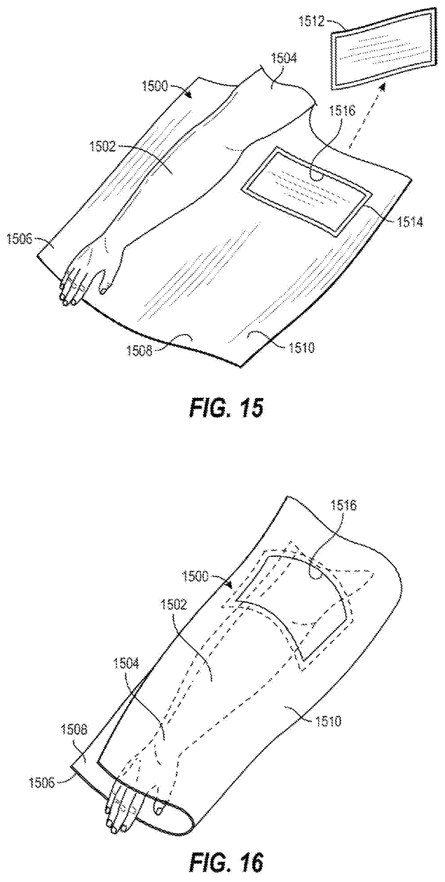

FIGS. 15-17 illustrate a medical bandage 1500 in use. FIG. 15 illustrates the medical bandage 1500 laid open and receiving and underlying an arm 1502 of a patient 1504 on a first portion 1506 of a bandage substrate 1508. A second portion 1510 of the bandage substrate extends along the side the arm 1502, presenting an inner protective cover 1512 that is removed to expose an inner adhesive layer 1514 around a window opening 1516 formed in the second portion 1510.

FIG. 16 illustrates the medical bandage 1500 wrapped about the arm 1502 of the patient 1504, placing the window opening 1516 over the forearm of the patient, defining an area of the arm 1502 that is sterilized. The rest of the covered arm 1502 can remain unsterilized without posing a contamination risk to the forearm used for accessing a blood vein.

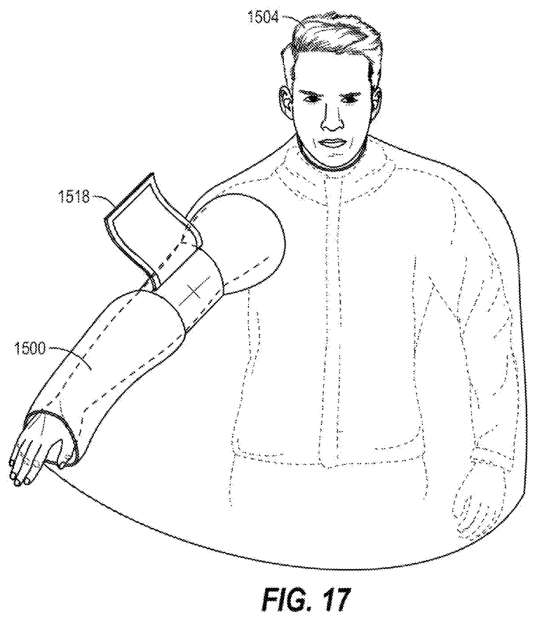

FIG. 17 illustrates the patient 1504 wearing the medical bandage 1500 with an outer protective cover 1518 opened to expose the forearm.

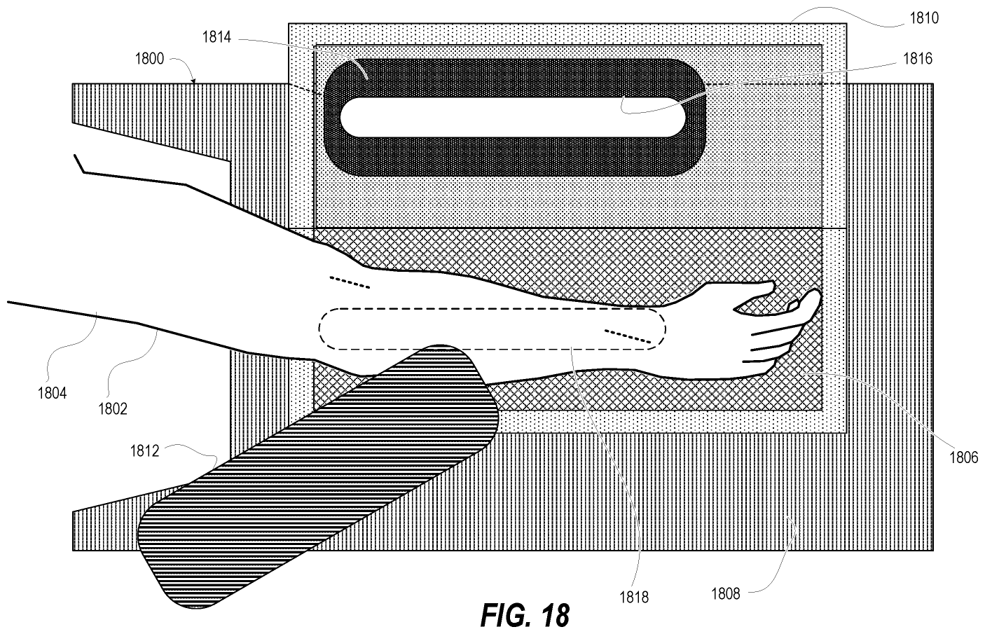

FIG. 18 illustrates a top view of an example single-access forearm medical bandage/surgical drape 1800 in an open configuration and receiving an underlying arm 1802 of a patient 1804 on an underlying first portion 1806 of a bandage substrate 1808.

A second portion 1810 of the bandage substrate extends along the side of the arm 1802, presenting a removable inner protective cover 1812 that is removed to expose an inner adhesive layer 1814 around a window opening 1816 formed in the second portion 1810. The second portion 1810 is attached as a flap to the first portion 1806. The upper surface of the arm 1802 is positioned to receive the adhesive layer 1814 around an area of interest 1818 that is intended to be kept sterile.

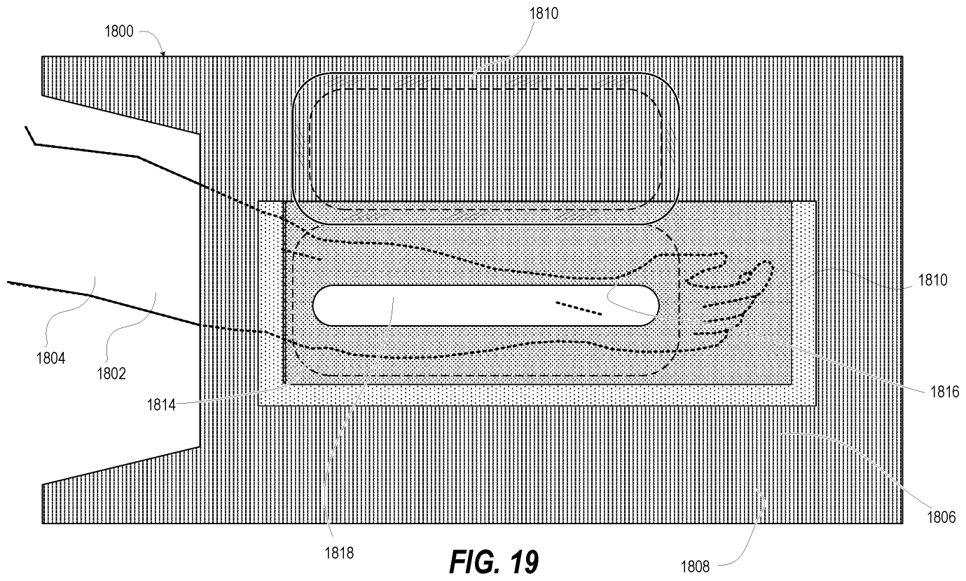

FIG. 19 illustrates a top view of the example single-access forearm medical bandage 1800 in a closed configuration. An outer protective cover 1820 is transparent to view an enclosed arm 1802. The outer protective cover 1820 is hingedly attached as a flap to the second portion 1810 to allow access to the area of interest 1818 for sterilizing and inserting intravenous needles. An inner edge of the outer protective cover 1820 can include a releasable adhesive that seals around window opening 1816.

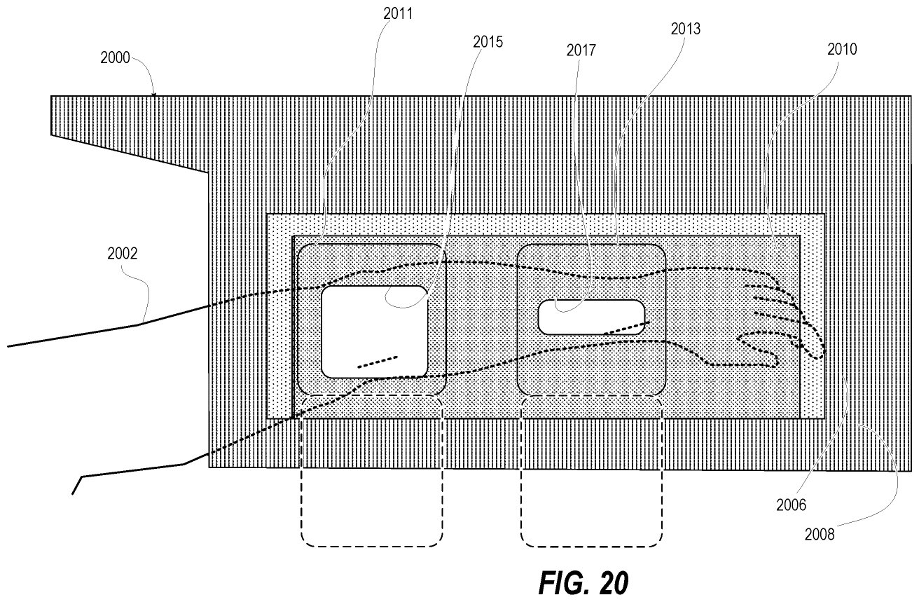

FIG. 20 illustrates a top view of an example double-access forearm medical bandage 2000 in a closed configuration with an arm 2002 resting on an underlying first portion 2006 of a bandage substrate 2008. Either or both of a midarm and a wrist outer protective cover 2011, 2013 can be opened respective windows 2015, 2017 formed in the second portion 2010 to expose respective sites of blood veins.

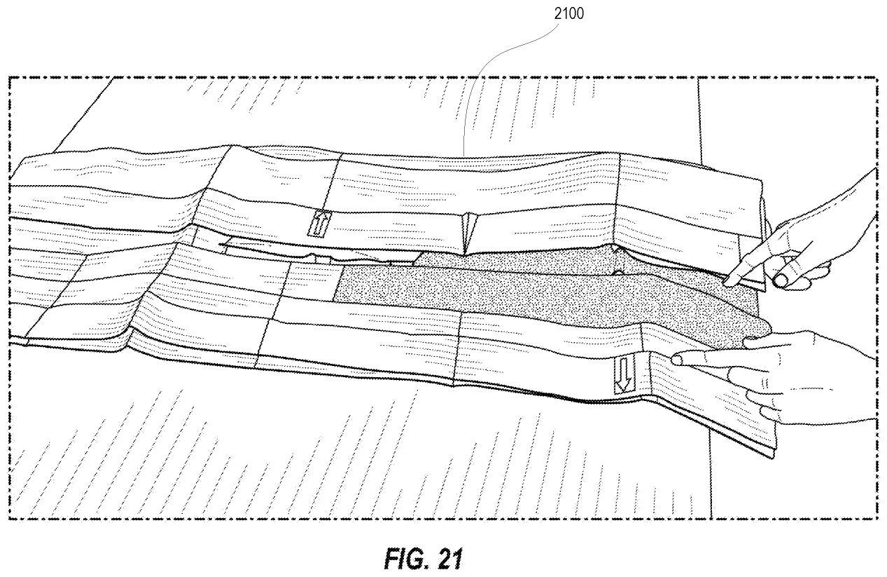

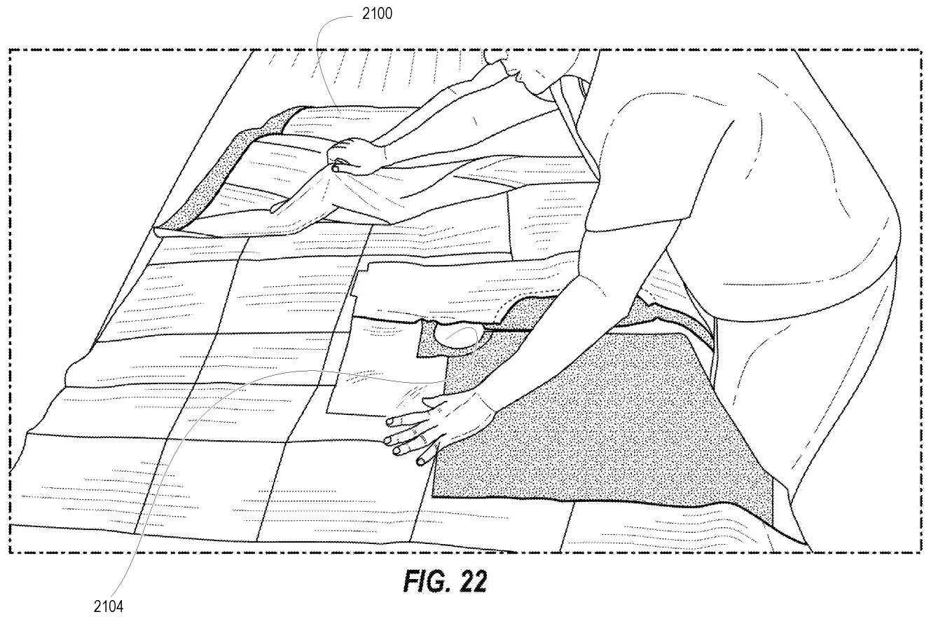

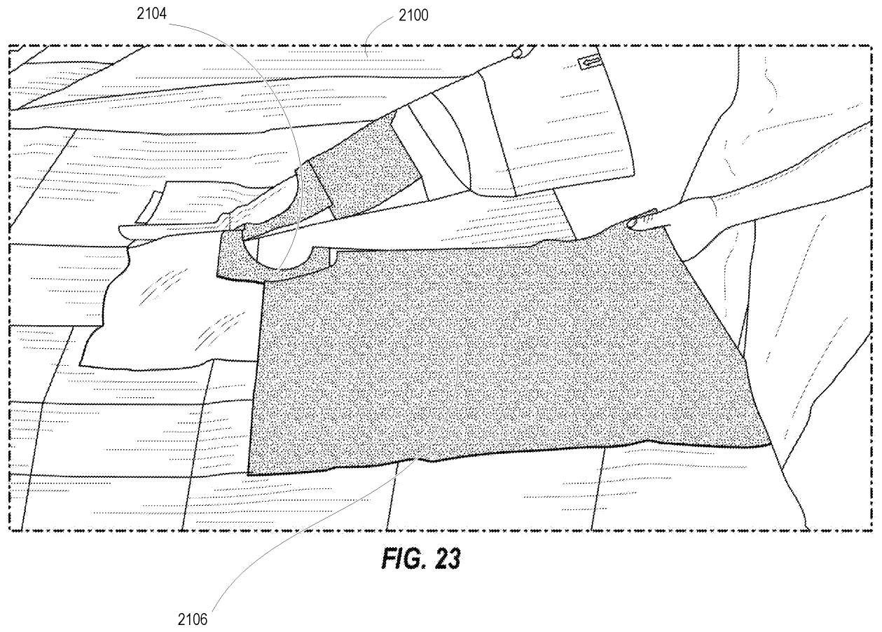

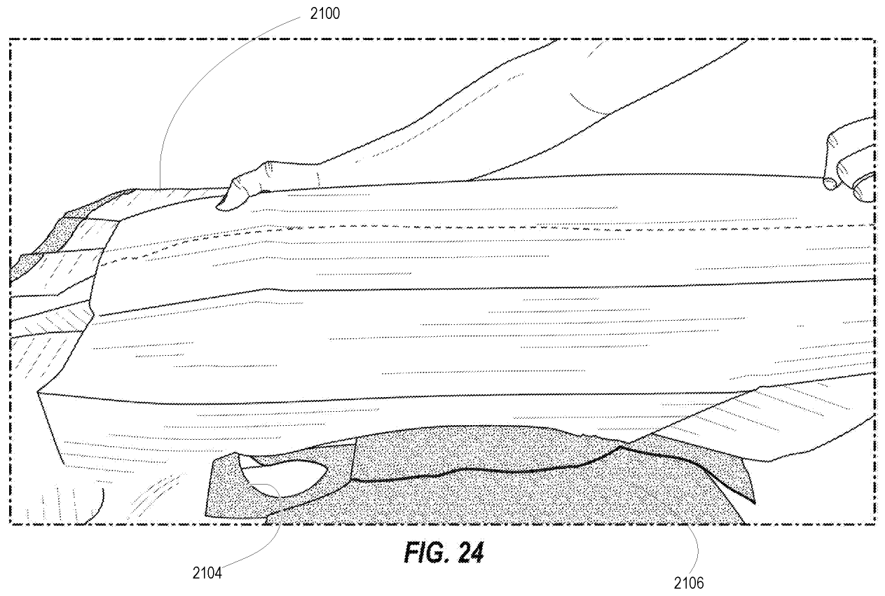

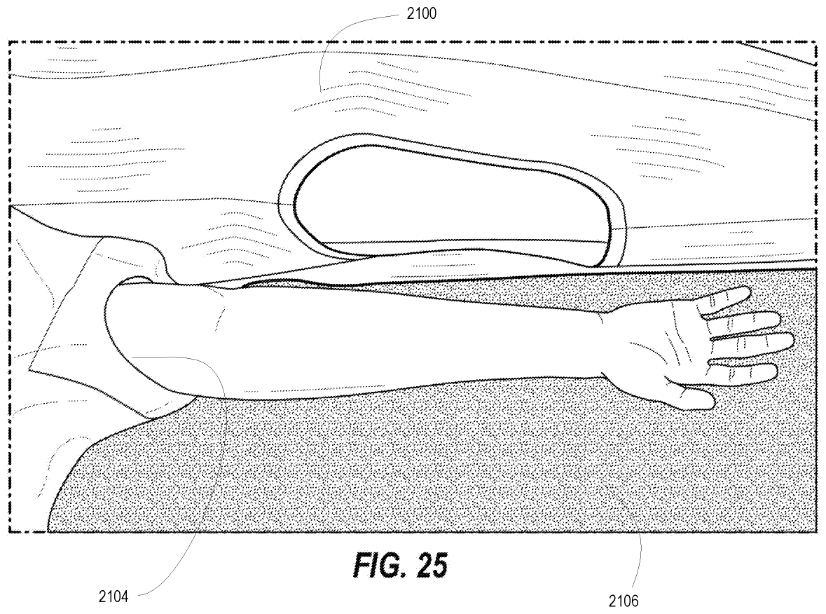

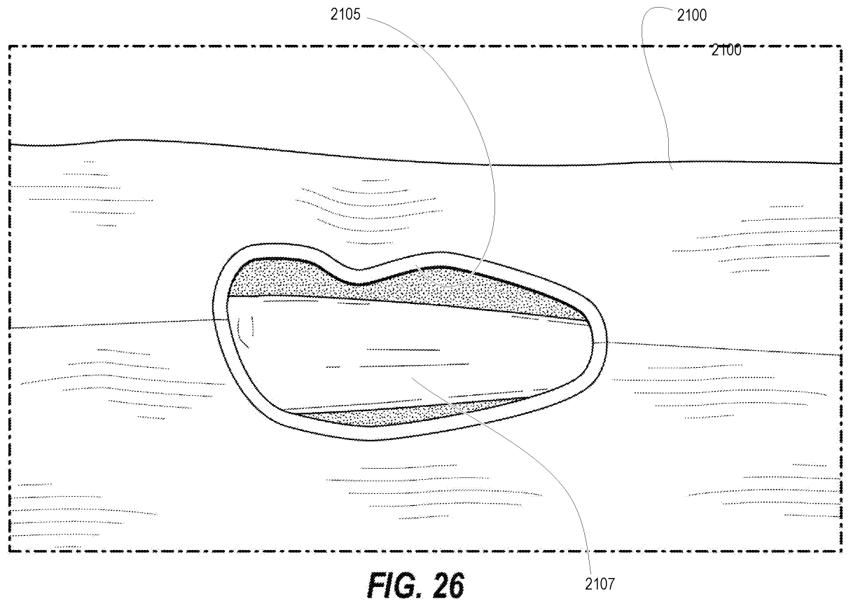

FIGS. 21-26 are the views of a medical bandage 2100 having sufficient size to serve as a gown in the one version. FIG. 21 illustrates a folded medical bandage 2100, according to one or more embodiments. Medical bandage 2100 is folded so that someone can grab the folded gown at two indicated points and pull apart then grab in two other points (90 degrees) and pull to fully extend. FIG. 22 illustrates an unfolded medical bandage 2100, according to one or more embodiments. The bandage could be made in a small or large version. The small version has a slit 2104 for putting the arm or leg through and then covers half the body. A second drape covers the rest. The large version is made to put an arm or leg through the slit 2104 and then cover the whole body. In one or more embodiments, medical bandage 2100 is scaled to either an arm version or a leg version given the differences. FIG. 23 illustrates the unfolded medical bandage 2100 with a slit 2104 that is sized for a shoulder opening, according to one or more embodiments. A nonsterile landing section 2106 is adjacent to the slit 2104 for receiving the arm or leg. Nonsterile landing section 2106 has a pad for absorbing fluids. FIG. 24 illustrates a forearm portion of the unfolded medical bandage 2100, according to one or more embodiments. FIG. 25 illustrates a forearm placed on the forearm portion of the unfolded medical bandage 2100, according to one or more embodiments. FIG. 26 illustrates the forearm covered by the medical bandage 2100, according to one or more embodiment. Window opening 2105 exposes a length of an arm 2107. In one or more embodiments, the landing section may include a padded section. The padded section can include any number of wound covering materials known in the art and can include an absorbent material, a coating or membrane configured to prevent clotted blood from sticking to the padding, medication or a treated coating, and any other materials or substances known in the art for use upon a bandage applied to a wound. In another exemplary embodiment, the landing section may include one or more replaceable padding portions and padded section. In one embodiment, the added padded section is thicker and can be more absorbent, for example, so that a medicated liquid, powder, or ointment can be applied to the wound.

The surgical bandage/drape may be equipped with factory-made or pre-made perforations. These perforations may be presented in various patterns so that the surgical drape may be configured for use by different surgeons or for different types of procedures. These perforations provide the surgeon the option of selecting (1) the size of the drape to be used in the operation and (2) the size of the surgical field. Different operations require various sizes of surgical drapes and sterile fields. By tailoring the size of the surgical drape to the operation or procedure, the surgeon can reduce the risk of dislodging the drape and compromising any sterile field.

The surgical bandage/drape may also or alternatively be equipped with markings; the markings, used either in conjunction with or instead of the factory perforations, will allow the surgeon to more accurately line up the patient's skin during the closure of the surgical site. This feature will allow for a more aesthetic and symmetrical closing, therefore minimizing the chances for any type of disfigurement to the patient. With the current invention in place, the surgeon can utilize the markings, or perforations, on the surgical drape to guide alignment and complete the closure with little worry as to symmetry or positioning.

Due to the many ways the current invention can be utilized, a “surgical kit” may be assembled for retail sale. This kit would include not only the aforementioned surgical bandage/drape but will also include the surgical tape and/or surgical glue discussed above as part of a more aesthetic closing technique. The surgical tape may be made of a similar material to the surgical drape, or a currently used paper tape, and would adhere to the surgical drape more strongly than to the skin. Usage of this would allow for “cross-taping” over the incision where the surgical drape was left on post-operatively, therefore increasing the chances that the closure can be made suture or staple free yet retain the time advantage of staples without the physical scarring associated with them. The surgical glue would adhere the skin to itself, eliminating the need for top layer suturing or stapling. It may be provided in a single use applicator packaged in a blister pouch. The applicator may be a crushable glass ampule contained within a plastic vial with attached applicator tip. The surgical glue provides the same benefits as the tape, where closure and/or scarring reduction is concerned. Providing a variety of surgically useful devices in one surgical kit not only provides convenience for the surgeon and his staff but would also aid in the surgeon's ability to provide better patient care.

In one or more embodiments, methods may be embodied in an automated system that performs a series of functional processes. In some implementations, certain steps of the methods are combined, performed simultaneously or in a different order, or perhaps omitted, without deviating from the scope of the disclosure. Thus, while the method blocks are described and illustrated in a particular sequence, use of a specific sequence of functional processes represented by the blocks is not meant to imply any limitations on the disclosure. Changes may be made with regards to the sequence of processes without departing from the scope of the present disclosure. Use of a particular sequence is therefore, not to be taken in a limiting sense, and the scope of the present disclosure is defined only by the appended claims.

It must be noted that, as used in this specification and the appended claims, the singular forms “a,” “an” and “the” include plural referents unless the content clearly dictates otherwise. Thus, for example, reference to a “colorant agent” includes two or more such agents.

Unless defined otherwise, all technical and scientific terms used herein have the same meaning as commonly understood by one of ordinary skill in the art to which the invention pertains. Although a number of methods and materials similar or equivalent to those described herein can be used in the practice of the present innovation, the exemplary materials and methods are described herein.

As will be appreciated by one having ordinary skill in the art, the methods and compositions of the invention substantially reduce or eliminate the disadvantages and drawbacks associated with prior art methods and compositions.

It should be noted that, when employed in the present disclosure, the terms “comprises,” “comprising,” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

As required, detailed embodiments of the present innovation are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present innovation in virtually any appropriately detailed structure.

While it is apparent that the illustrative embodiments of the invention herein disclosed fulfill the objectives stated above, it will be appreciated that numerous modifications and other embodiments may be devised by one of ordinary skill in the art. Accordingly, it will be understood that the appended claims are intended to cover all such modifications and embodiments, which come within the spirit and scope of the present innovation.

What is claimed is:

1. A medical bandage, comprising:
a bandage substrate having a portion designated to receive and underlie an arm of a patient as a non-sterile area;
a flap attached longitudinally along one edge of the non-sterile area of the bandage substrate and positionable in: (i) an open position for insertion of the arm of the patient; and (ii) a closed position over top of the arm of the patient, the flap having an opening that exposes at least a forearm portion as a sterilizable area of the arm of the patient for therapeutic or diagnostic access to a blood vein;
an inner adhesive layer attached to an inner surface of the flap around the opening to releasably adhere to the arm of the patient;
an access panel sized to cover the opening; and
an outer adhesive layer attached to an outer surface of the flap around the opening to releasably adhere to an outer annular area of the access panel.

2. The medical bandage of claim 1, further comprising a removable backing layer selectively removable from the inner adhesive layer to expose the inner adhesive layer.

3. The medical bandage of claim 1, further comprising a neck support portion positionable around the neck of the patient.

4. The medical bandage of claim 1, further comprising a shoulder support portion positionable over the opposite shoulder of the patient.

5. A medical bandage, comprising:
a bandage substrate having a first portion designated to receive and underlie an arm of a patient as a non-sterile area and having a second portion that wraps over a forearm including between a wrist and an inner side of the elbow of the patient, the second portion having an opening that exposes at least a portion of the forearm for accessing a blood vein;
an inner adhesive layer attached around a periphery of the second portion around the opening to adhere to the forearm of the patient, separating an unsterilized portion of the patient's arm from a sterilized portion of the patient's arm exposed through the opening; and
an outer protective cover releasably engaged to the close the opening.

6. The medical bandage of claim 5, further comprising an inner protective cover releasably attached to the inner adhesive layer that is removable prior to use.

7. The medical bandage of claim 5, wherein the outer protective cover comprises a flap attached along one edge to the bandage substrate.

8. The medical bandage of claim 5, wherein the outer protective cover is transparent.
